(19) United States
(12) Patent Application Publication
(10) Pub. No.: US 2021/0264618 A1
(43) Pub. Date: Aug. 26, 2021

(54) EYE MOVEMENT MEASUREMENT DEVICE, EYE MOVEMENT MEASUREMENT METHOD, AND EYE MOVEMENT MEASUREMENT PROGRAM

(71) Applicant: University of Tsukuba, Ibaraki (JP)

(72) Inventors: Kiyoshi Hoshino, Tsukuba-shi (JP); Nayuta Ono, Tokyo (JP)

(21) Appl. No.: 16/973,754

(22) PCT Filed: Jun. 12, 2019
(86) PCT No.: PCT/JP2019/023226

§ 371 Date: Dec. 9, 2020

(30) Foreign Application Priority Data
Jun. 12, 2018 (JP) 2018-111535

(51) Int. Cl. G06T 7/246; G06K 9/00; G06K 9/20; A61B 3/113; A61B 3/14; A61B 3/00

(52) U.S. Cl. CPC G06T 7/246; G06K 9/00604; G06K 9/0061; G06T 2207/30041; A61B 3/113; A61B 3/14; A61B 3/0008; G06K 9/2027

(57) ABSTRACT

An eye movement measurement device includes an acquisition unit configured to acquire an eye image in which an image of an eye of a subject is captured, a feature point extraction unit configured to extract feature points in a white region of the eye included in the eye image acquired by the acquisition unit, a candidate region generation unit configured to generate, for each of the feature points extracted by the feature point extraction unit, a template candidate region, the template candidate region being a region including a pixel of each of the feature points, in the eye image, a selection unit configured to select, as a template region, the template candidate region including more of the feature points among a plurality of the template candidate regions generated by the candidate region generation unit, and a measurement unit configured to measure a three-dimensional eye movement including at least a rotation angle of the eye of the subject by tracking movement in the eye image acquired by the acquisition unit by using the template region selected by the selection unit.

### TECHNICAL FIELD

The present invention relates to an eye movement measurement device, an eye movement measurement method, and an eye movement measurement program.

This application claims priority based on JP 2018-111535 filed in Japan on Jun. 12, 2018, the contents of which are incorporated herein by reference.

### BACKGROUND ART

Conventionally, techniques have been disclosed for measuring eye movement by capturing an image of an eye to quantify video sickness or 3D sickness, for example, caused by a virtual-reality (VR) image when using a head-mounted display (HMD) (see, for example, Patent Document 1).

### CITATION LIST

### Patent Literature

Patent Document 1: JP 2017-189470 A

### SUMMARY OF INVENTION

### Technical Problem

However, according to the techniques as that disclosed in Patent Document 1, there is a problem in that the measurement accuracy of eye movement (in particular, the eye rotation movement) may be reduced in some states of the captured image of the eye, and in this case, stable measurement cannot be achieved.

### Solution to Problem

An eye movement measurement device according to an embodiment of the present invention includes: an acquisition unit configured to acquire an eye image in which an image of an eye of a subject is captured, a feature point extraction unit configured to extract feature points in a white region of the eye included in the eye image acquired by the acquisition unit, a candidate region generation unit configured to generate, for each of the feature points extracted by the feature point extraction unit, a template candidate region, the template candidate region being a region including a pixel of each of the feature points, in the eye image, a selection unit configured to select, as a template region, the template candidate region including more of the feature points among a plurality of the template candidate regions generated by the candidate region generation unit, and a measurement unit configured to measure a three-dimensional eye movement including at least a rotation angle of the eye of the subject by tracking movement in the eye image acquired by the acquisition unit by using the template region selected by the selection unit.

In the eye movement measurement device according to an embodiment of the present invention, the candidate region generation unit generates the template candidate region for each of the feature points each selected as a feature point corresponding to a position of a blood vessel in the white region of the eye among the feature points extracted by the feature point extraction unit.

In the eye movement measurement device according to an embodiment of the present invention, the feature point extraction unit extracts the feature points in the white region of the eye by performing a statistical process including at least histogram equalization on pixel values of the respective pixels in the white region of the eye.

In the eye movement measurement device according to an embodiment of the present invention, the selection unit selects, as the template region, the template candidate region that less frequently matches a plurality of regions differing from each other in the eye image among a plurality of the template candidate regions.

The eye movement measurement device according to an embodiment of the present invention further includes an image capturing unit configured to capture an image of the eye of the subject to generate the eye image.

The eye movement measurement device according to an embodiment of the present invention further includes a first irradiation unit configured to irradiate the eye of the subject with electromagnetic waves of a wavelength longer than 570 nanometers, a second irradiation unit configured to irradiate the eye of the subject with electromagnetic waves of a wavelength shorter than 570 nanometers, and an irradiation control unit configured to cause one of the first irradiation unit and the second irradiation unit to emit electromagnetic waves.

An eye movement measurement method according to an embodiment of the present invention includes: acquiring an eye image in which an image of an eye of a subject is captured; extracting feature points in a white region of the eye included in the eye image acquired in the acquiring an eye image; generating, for each of the feature points extracted in the extracting feature points, a template candidate region, the template candidate region being a region including a pixel of each of the feature points, in the eye image; selecting, as a template region, the template candidate region including more of the feature points among a plurality of the template candidate regions generated in the generating a template candidate region; and measuring a three-dimensional eye movement including at least a rotation angle of the eye of the subject by tracking movement in the eye image acquired in the acquiring an eye image by using the template region selected in the selecting the template candidate region.

An eye movement measurement program according to an embodiment of the present invention for causing a computer to perform: acquiring an eye image in which an image of an eye of a subject is captured; extracting feature points in a white region of the eye included in the eye image acquired in the acquiring an eye image; generating, for each of the feature points extracted in the extracting feature points, a template candidate region, the template candidate region being a region including a pixel of each of the feature points, in the eye image; selecting, as a template region, the template candidate region including more of the feature points among a plurality of the template candidate regions generated in the generating a template candidate region; and measuring a three-dimensional eye movement including at least a rotation angle of the eye of the subject by tracking movement in the eye image acquired in the acquiring an eye image by using the template region selected in the selecting the template candidate region.

### Advantageous Effects of Invention

According to the present invention, an eye movement measurement device, an eye movement measurement method, and an eye movement measurement program can be provided that can achieve the improved measurement accuracy of eye movement.

### BRIEF DESCRIPTION OF DRAWINGS

FIG. 1 is a diagram illustrating an example of a functional configuration of an eye movement measurement system according to the present embodiment.

FIG. 2 is a diagram illustrating an example of a relative position in a case of an image capturing angle of 40 degrees and a distance of 15 mm.

FIG. 3 is a diagram illustrating an example of a relative position in a case of an image capturing angle of 50 degrees and a distance of 15 mm.

FIG. 4 is a diagram illustrating an example of a relative position in a case of an image capturing angle of 60 degrees and a distance of 15 mm.

FIG. 5 is a diagram illustrating an example of a relative position in a case of an image capturing angle of 40 degrees and a distance of 20 mm.

FIG. 6 is a diagram illustrating an example of a relative position in a case of an image capturing angle of 50 degrees and a distance of 20 mm.

FIG. 7 is a diagram illustrating an example of a relative position in a case of an image capturing angle of 60 degrees and a distance of 20 mm.

FIG. 8 is a diagram illustrating an example of a relative position in a case of an image capturing angle of 40 degrees and a distance of 25 mm.

FIG. 9 is a diagram illustrating an example of a relative position in a case of an image capturing angle of 50 degrees and a distance of 25 mm.

FIG. 10 is a diagram illustrating an example of a relative position in a case of an image capturing angle of 60 degrees and a distance of 25 mm.

FIG. 11 is a diagram illustrating an example of operation of an eye movement measurement system according to the present embodiment.

FIG. 12 is a diagram illustrating an example of operation of determining a template region of the eye movement measurement system according to the present embodiment.

FIG. 13 is a diagram illustrating an example of an eye image according to the present embodiment.

FIG. 14 is a diagram illustrating an example of an image of a white region of an eye after a histogram equalization process according to the present embodiment.

FIG. 15 is a diagram illustrating an example of an extraction result of feature points according to the present embodiment.

FIG. 16 is a diagram illustrating an example of a blood vessel binarized thinned image according to the present embodiment.

FIG. 17 is a diagram illustrating an example of blood vessel corresponding feature points according to the present embodiment.

FIG. 18 is a diagram illustrating an example of template candidate regions according to the present embodiment.

FIG. 19 is a diagram illustrating a modified example of a functional configuration of an eye movement measurement system.

### DESCRIPTION OF EMBODIMENTS

### Embodiments

An eye movement measurement system 1 according to the present embodiment will be described below with reference to the drawings.

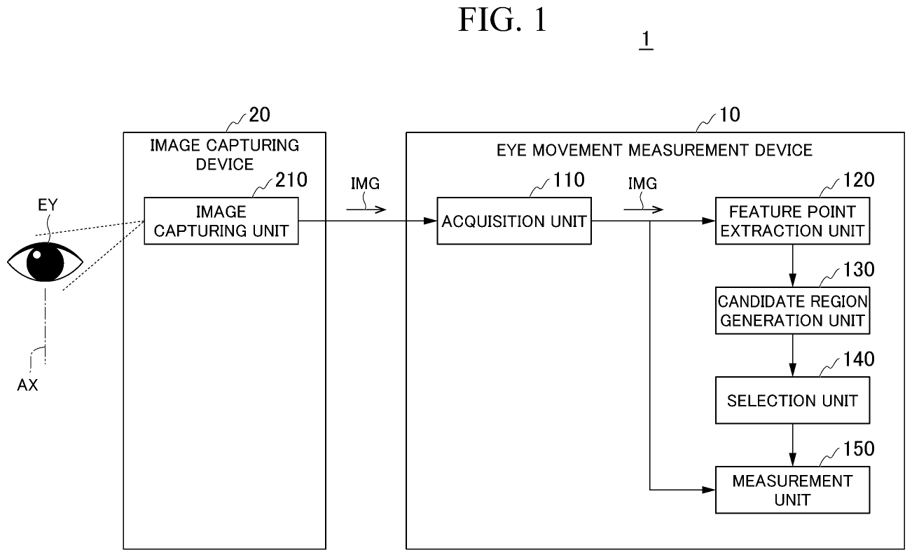

FIG. 1 illustrates an example of a functional configuration of the eye movement measurement system 1 according to the present embodiment. The eye movement measurement system 1 includes an eye movement measurement device 10 and an image capturing device 20.

Note that in this example, a case in which the eye movement measurement device 10 and the image capturing device 20 are configured as different devices will be described, but no such limitation is intended. The eye movement measurement device 10 and the image capturing device 20 may be configured as one integrated device.

First, a configuration of the image capturing device 20 will be described, and next, a configuration of the eye movement measurement device 10 will be described.

### Functional Configuration of Image Capturing Device 20

The image capturing device 20 includes an image capturing unit 210. The image capturing unit 210 includes a camera capable of capturing a moving image, for example. The image capturing unit 210 generates an eye image IMG by capturing an image of an eye EY of a subject SB.

In this example, the image capturing device 20 is configured as spectacle-type goggles mounted on the head of the subject SB. The image capturing device 20 includes a color board camera for capturing an image of blood vessels as the image capturing unit 210, and captures a blood vessel image and an image of the pupil of the eye EY. The color board camera is installed at the same height as the eye EY at a position separated 20 mm (millimeters) from the eye in a 50-degree direction from the front toward the outer corner of the eye, and mainly captures an image of the iris of the eye EY and a white region EW of the eye located closer to the outer corner falling within the angle of view. The screen resolution of the image capturing unit 210 is 720×480 [pixels], and the image capturing speed is 29.97 [fps].

Here, a relative positional relationship between the eye EY and the image capturing unit 210 will be described.

### Relative Positional Relationship Between Eye and Image Capturing Unit

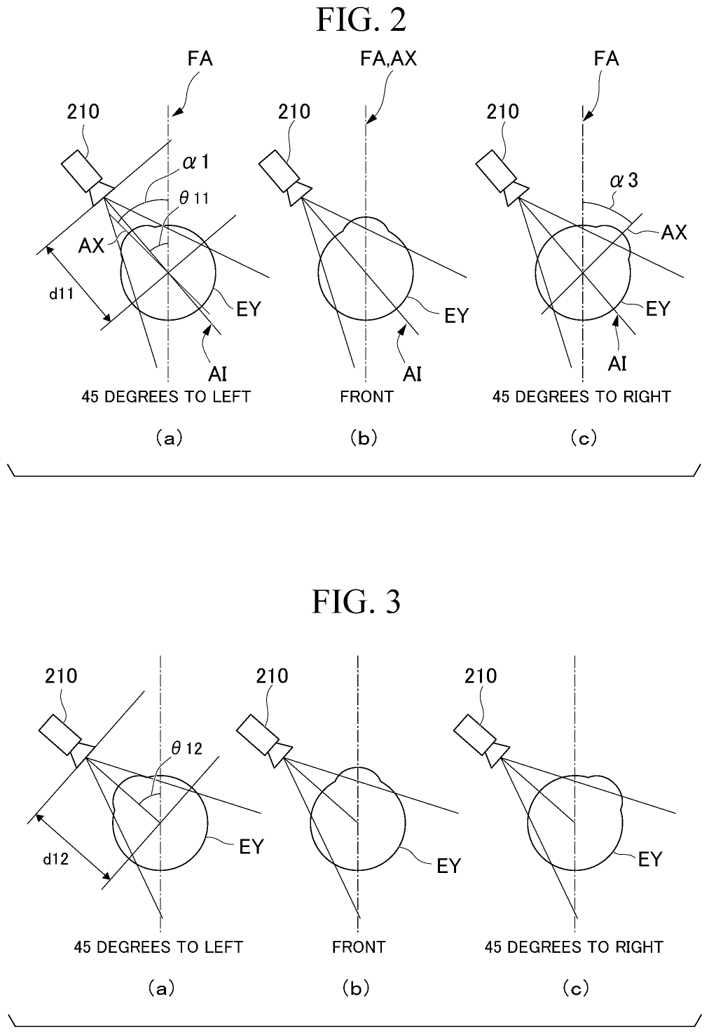

FIGS. 2 to 10 are diagrams each illustrating an example of a relative positional relationship between the eye EY and the image capturing unit 210. In the following description, the angle formed by the direction (front direction FA) of the head front of the subject SB and the direction of the line-of-sight axis AX of the eye EY is referred to as an “angle α”, and the angle formed by the front direction FA and the direction of the image capturing axis AI of the image capturing unit 210 is referred to as an “angle θ”. Note that in the following description, the angle α is also referred to as a “line-of-sight angle α”, and the angle θ is also referred to as an “image capturing angle θ”. A distance between the lens of the image capturing unit 210 and the center of the eye EY is referred to as a “distance d”.

First, a case in which the distance d is 15 mm will be described.

FIG. 2 is a diagram illustrating an example of a relative positional relationship in a case where an image capturing angle θ11 (40 degrees) and a distance d11 (15 mm) are used.

In FIG. 2(a), the direction of a line-of-sight axis AX of the eye EY is rotated 45 degrees counterclockwise with respect to a front direction FA. In other words, an angle α1, namely, the line-of-sight angle α, is 45 degrees. In FIG. 2(a), the subject SB looks in the left direction while turning their head forward in front of them.

In this case, the angle of view of the image capturing unit 210 includes both the white portion of the eye EY and the iris portion of the eye EY. In other words, in the state illustrated in FIG. 2(a), the image capturing unit 210 can capture an image of the white portion of the eye EY and the iris portion of the eye EY.

In FIG. 2(b), the line-of-sight axis AX of the eye EY is aligned with the direction of the front direction FA, and the line-of-sight angle α is 0 degrees. In other words, in FIG. 2(b), the subject SB looks forward in front of them. In the case of this example, the angle of view of the image capturing unit 210 includes both the white portion of the eye EY and the iris portion of the eye EY. In other words, in the state illustrated in FIG. 2(b), the image capturing unit 210 can capture an image of the white portion of the eye EY and the iris portion of the eye EY.

In FIG. 2(c), the direction of the line-of-sight axis AX of the eye EY is rotated 45 degrees clockwise with respect to the front direction FA. In other words, the angle α3 is 45 degrees. In FIG. 2(c), the subject SB looks in the right direction while turning their head forward in front of them. In this case, the angle of view of the image capturing unit 210 includes the white portion of the eye EY, but does not include the iris portion of the eye EY. In other words, in the state illustrated in FIG. 2(c), the image capturing unit 210 can capture an image of the white portion of the eye EY, but cannot capture an image of the iris portion of the eye EY.

Note that in the following description, portions in which the positional relationship between the image capturing unit 210 and the eye EY are similar to those illustrated in FIG. 2 will be omitted.

FIG. 3 is a diagram illustrating an example of a relative positional relationship in a case where an image capturing angle θ12 (50 degrees) and a distance d12 (15 mm) are used. In the state of FIG. 3(a), the image capturing unit 210 can capture both the images of the white of the eye and the iris of the eye. In the state of FIG. 3(b), the image capturing unit 210 can capture both the images of the white of the eye and the iris of the eye. In the state of FIG. 3(c), the image capturing unit 210 can capture the image of the white of the eye, but cannot capture the image of the iris of the eye.

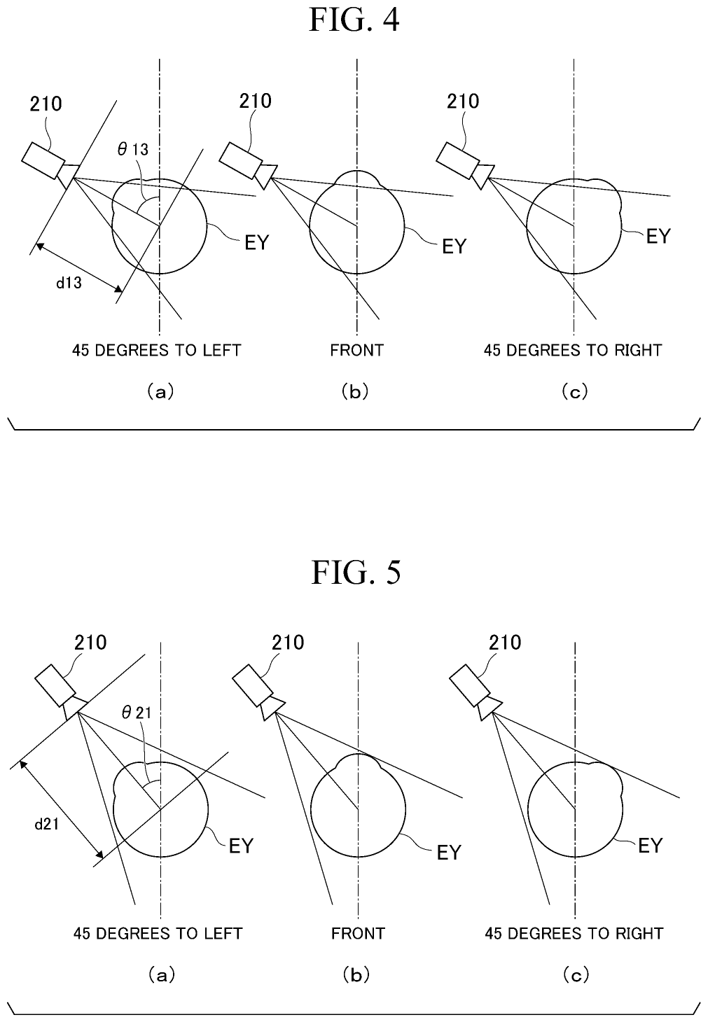

FIG. 4 is a diagram illustrating an example of a relative positional relationship in a case where an image capturing angle θ13 (60 degrees) and a distance d13 (15 mm) are used. As illustrated in FIG. 4, a case where the images of the white of the eye and iris of the eye can be captured is the same as the case described with reference to FIGS. 2 and 3.

In other words, in a case where the distance d is 15 mm, depending on the direction of the line-of-sight axis AX, the images of the white of the eye and the iris of the eye may not captured at the same time.

Next, a case in which the distance d is 20 mm will be described.

FIG. 5 is a diagram illustrating an example of a relative positional relationship in a case where an image capturing angle θ21 (40 degrees) and a distance d21 (20 mm) are used.

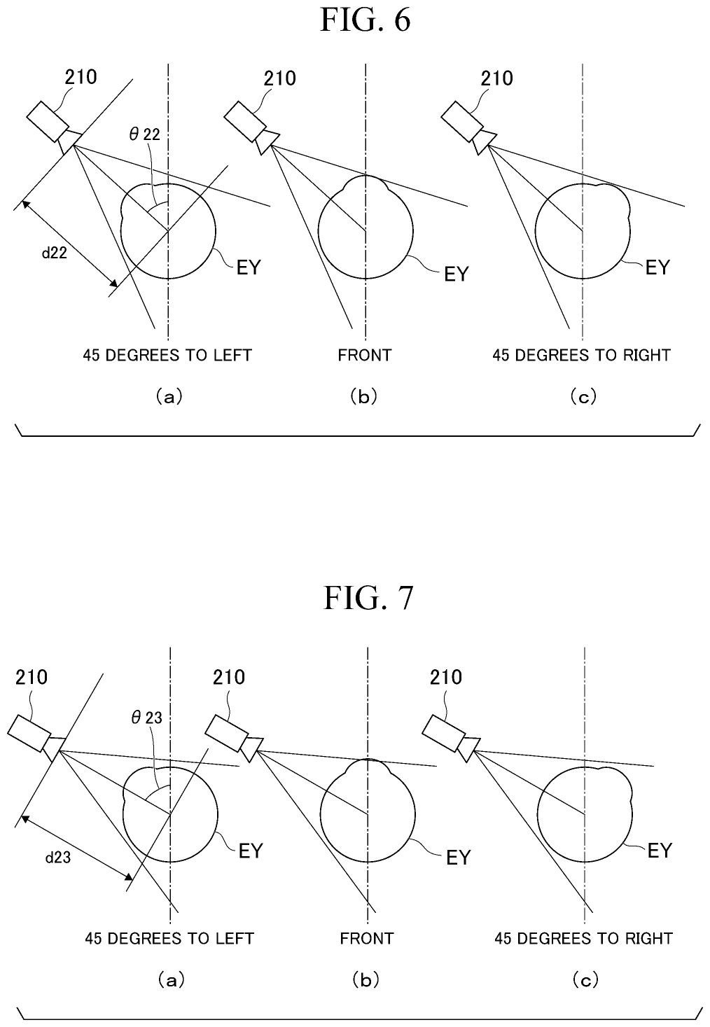

FIG. 6 is a diagram illustrating an example of a relative positional relationship in a case where an image capturing angle θ22 (50 degrees) and a distance d22 (20 mm) are used.

FIG. 7 is a diagram illustrating an example of a relative positional relationship in a case where an image capturing angle θ23 (60 degrees) and a distance d23 (20 mm) are used.

According to FIGS. 5 to 7, in a case where the image capturing angle θ is the image capturing angle θ23 (60 degrees), the images of the white of the eye and the iris of the eye may not be captured at the same time. On the other hand, in a case where the image capturing angle θ is the image capturing angle θ21 (40 degrees) and the image capturing angle θ22 (50 degrees), the images of the white of the eye and the iris of the eye can be captured at the same time regardless of the direction of the line-of-sight axis AX.

Next, a case in which the distance d is 25 mm will be described.

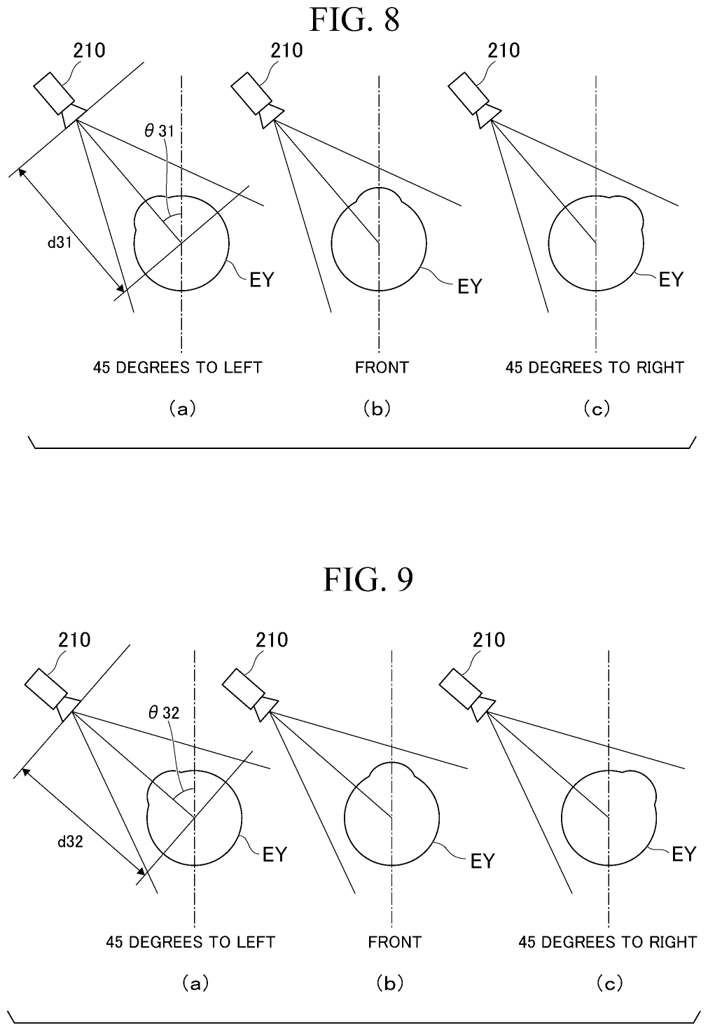

FIG. 8 is a diagram illustrating an example of a relative positional relationship in a case where an image capturing angle θ31 (40 degrees) and a distance d31 (25 mm) are used.

FIG. 9 is a diagram illustrating an example of a relative positional relationship in a case where an image capturing angle θ32 (50 degrees) and a distance d32 (25 mm) are used.

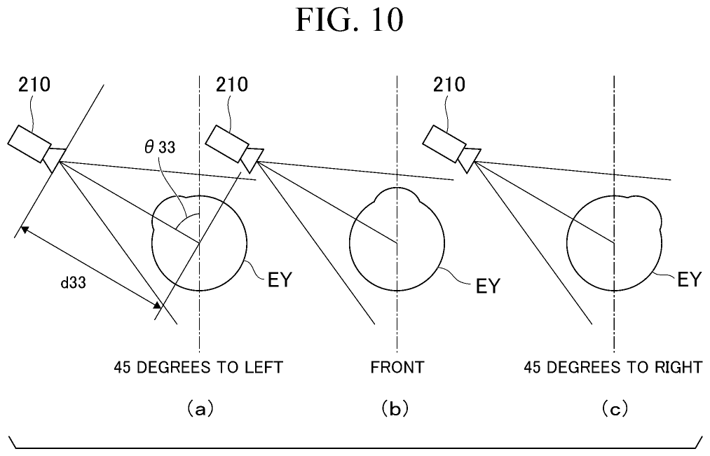

FIG. 10 is a diagram illustrating an example of a relative positional relationship when an image capturing angle θ33 (60 degrees) and a distance d32 (25 mm) are used.

According to FIGS. 8 to 10, even though the image capturing angle θ is any of the image capturing angle θ31, the image capturing angle θ32, and the image capturing angle θ33, the images of the white of the eye and the iris of the eye can be captured at the same time regardless of the direction of the line-of-sight axis AX.

In other words, in a case where the relative positional relationship between the image capturing unit 210 and the eye EY changes, there may be a case where the images of the white of the eye and the iris of the eye can be captured at the same time, or a case where these cannot be captured at the same time. In this example, to capture the images of the white of the eye and the iris of the eye at the same time, the distance d is preferably large, and the image capturing angle θ is preferably small.

To increase the area of the white of the eye that falls within the angle of view of the image capturing unit 210, the distance d is preferably small, and the image capturing angle θ is preferably large. In other words, the relative positional relationship between the image capturing unit 210 and the eye EY is required to be within a predetermined range in order to increase the area of the white of the eye that falls within the angle of view of the image capturing unit 210 while the images of the white of the eye and the iris of the eye can be captured at the same time. As an example of this predetermined range, as described above, the distance d is preferably from 20 to 25 mm and the image capturing angle θ is preferably from 40 degrees to 50 degrees. As an example of this predetermined range, as described above, in a case where the distance d is 25 mm, the image capturing angle θ may be 60 degrees.

### Functional Configuration of Eye Movement Measurement Device 10

Returning to FIG. 1, the functional configuration of the eye movement measurement device 10 will be described. The eye movement measurement device 10 includes an acquisition unit 110, a feature point extraction unit 120, a candidate region generation unit 130, a selection unit 140, and a measurement unit 150.

The acquisition unit 110 acquires the eye image IMG in which the image of the eye EY of the subject SB is captured.

The feature point extraction unit 120 extracts feature points FP in a white region EW of the eye included in the eye image IMG acquired by the acquisition unit 110.

The candidate region generation unit 130 generates, for each feature point FP, a template candidate region TC, which is a region including the pixel of the feature point FP extracted by the feature point extraction unit 120, in the eye image IMG.

The selection unit 140 selects the template candidate region TC including more of the feature points FP among a plurality of template candidate regions TC generated by the candidate region generation unit 130, as the template region TP.

The measurement unit 150 measures the three-dimensional eye movement including at least a rotation angle AT of the eye EY of the subject SB by tracking the movement of the eye image IMG acquired by the acquisition unit 110 by using the template region TP selected by the selection unit 140.

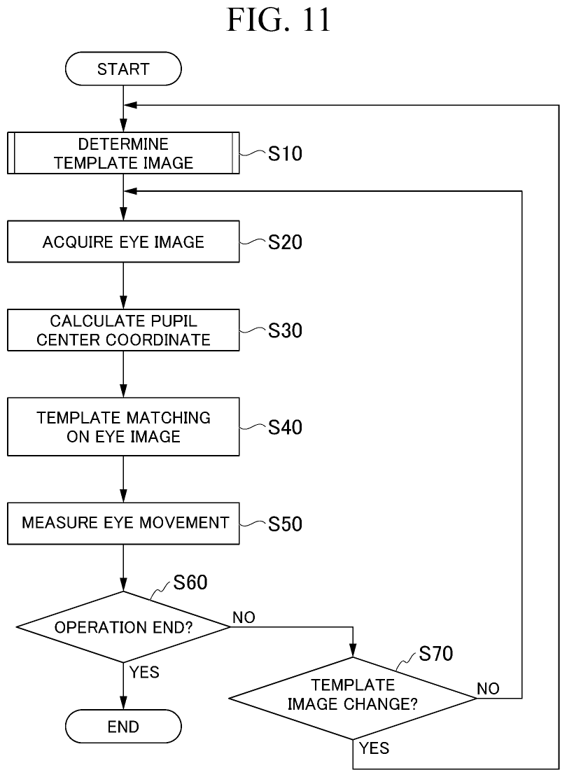

Specific examples of operations of these components will be described with reference to FIG. 11.

### Operation of Eye Movement Measurement System

FIG. 11 is a diagram illustrating an example of operation of the eye movement measurement system 1 according to the present embodiment.

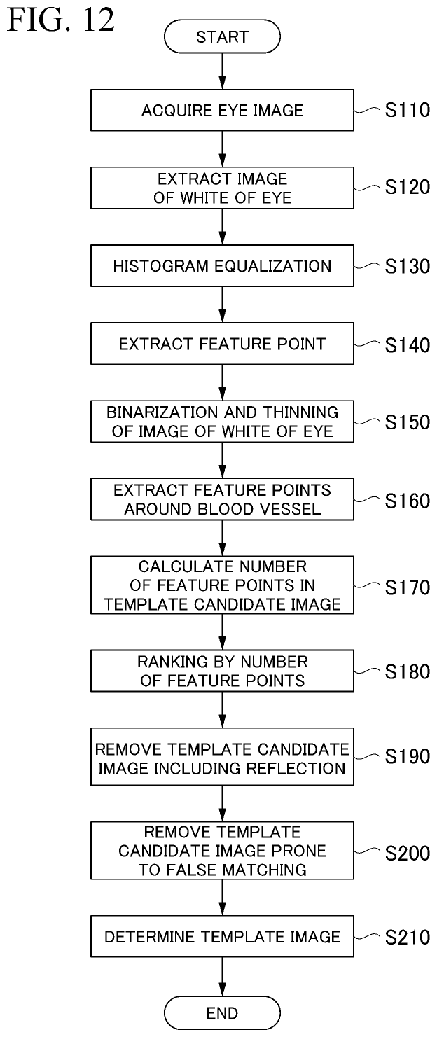

(Step S10) The eye movement measurement device 10 determines a template region TP. Here, details of the procedure in which the eye movement measurement device 10 determines the template region TP will be described with reference to FIG. 12.

### Determination of Template Image

FIG. 12 is a diagram illustrating an example of operation of determining the template region TP in the eye movement measurement system 1 according to the present embodiment.

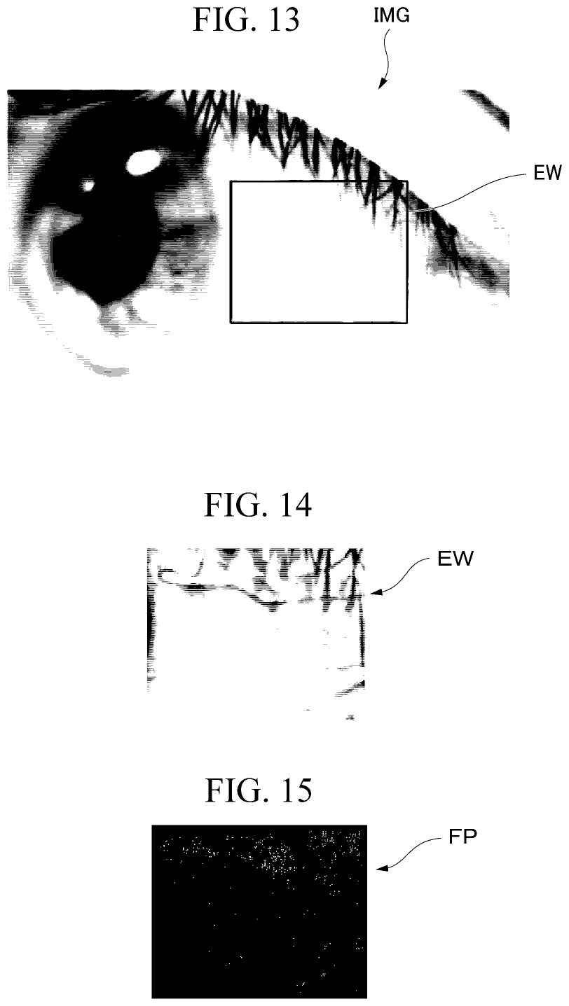

(Step S110) The acquisition unit 110 acquires the eye image IMG captured by the image capturing unit 210. An example of this eye image IMG is illustrated in FIG. 13.

FIG. 13 is a diagram illustrating an example of an eye image IMG according to the present embodiment. In this example, the image capturing unit 210 captures an image of the left eye EY of the subject SB and generates an eye image IMG. The eye image IMG includes the white region EW of the eye.

(Step S120) The feature point extraction unit 120 extracts an image (also referred to as an image of the white of the eye) of the white region EW of the eye from the eye image IMG acquired by the acquisition unit 110.

(Step S130) The feature point extraction unit 120 performs histogram equalization on the image of the white of the eye extracted in step S120. With this histogram equalization, the feature point extraction unit 120 emphasizes the image of blood vessels included in the white region EW of the eye by increasing the light and shade contrast between the white region EW of the eye and the blood vessel image.

Specifically, the feature point extraction unit 120 performs the conversion represented by in Equation (1) for a pixel value (for example, the luminance value) of each pixel of the eye image IMG.

$${z}^{′} = \frac{255}{Height \cdot Width} \cdot {\sum }_{i = 0}^{z} h ( z ) \qquad ( 1 )$$

Here, z is a luminance value before conversion, z′ is a luminance value after conversion, h(z) is the number of pixels in a luminance value z, Height is a vertical size of an input image, and Width is a horizontal size of the input image.

In other words, the feature point extraction unit 120 performs a statistical process including at least histogram equalization for the pixel value of each pixel in the white region EW of the eye. An example of an image of the white region EW of the eye after the feature point extraction unit 120 has performed histogram equalization is illustrated in FIG. 14.

FIG. 14 is a diagram illustrating an example of an image of the white region EW of the eye after the histogram equalization process according to the present embodiment.

(Step S140) Returning to FIG. 12, the feature point extraction unit 120 extracts the feature points FP by a known method (for example, oriented FAST and rotated BRIEF (ORB)) for the image of the white of the eye on which histogram equalization has performed. An example of the result of the feature point extraction unit 120 extracting the feature points FP is illustrated in FIG. 15.

FIG. 15 is a diagram illustrating an example of the extraction result of the feature points FP according to the present embodiment. The feature point extraction unit 120 extracts the feature points FP in the white region EW of the eye by performing a statistical process on the pixel value of each pixel in the white region EW of the eye. In this example, the feature point extraction unit 120 performs histogram equalization as the statistical process for each pixel.

(Step S150) Returning to FIG. 12, the feature point extraction unit 120 binarizes the image of the white of the eye extracted in step S120, and further generates an image (blood vessel binarized thinned image BTN) obtained by thinning the binarized image. Specifically, the feature point extraction unit 120 performs an adaptive binarization process in which the numeric value obtained by subtracting an offset value (for example, 4) from the sum of the luminance values weighted by Gaussian for the size 17×17 [pixels] of a nearby region is used as a threshold, and then performs the thinning process. As a result, a position PV of each blood vessel included in the image of the white region EW of the eye is extracted.

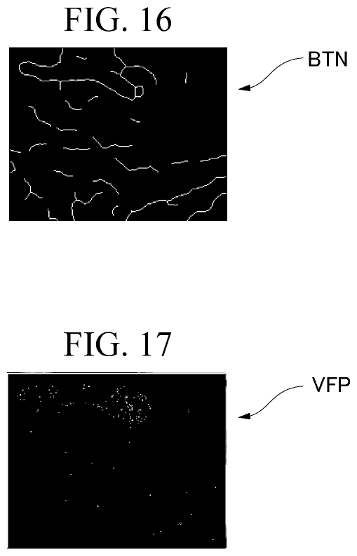

An example of the blood vessel binarized thinned image BTN generated by the feature point extraction unit 120 is illustrated in FIG. 16.

FIG. 16 is a diagram illustrating an example of the blood vessel binarized thinned image BTN according to the present embodiment.

(Step S160) Returning to FIG. 12, the feature point extraction unit 120 extracts the feature points (blood vessel corresponding feature points VFP) surrounding the blood vessels among the feature points FP extracted in step S140, by superimposing the feature points FP extracted in step S140 and the position PV of each blood vessel extracted in step S150. An example of the blood vessel corresponding feature points VFP extracted by the feature point extraction unit 120 is illustrated in FIG. 17.

FIG. 17 is a diagram illustrating an example of the blood vessel corresponding feature points VFP according to the present embodiment. In other words, the feature point extraction unit 120 selects the blood vessel corresponding feature points VFP as the feature points FP each corresponding to the position PV of each blood vessel in the white region EW of the eye among the feature points FP.

(Step S170) Returning to FIG. 12, the candidate region generation unit 130 counts how many feature points are included in a region centered on a certain feature point FP among the feature points FP extracted in step S140 (for example, a region of 50 [pixels]×50 [pixels]) for each of the feature points extracted in step S140. In the following description, a region centered on a feature point FP is also referred to as a template candidate region TC.

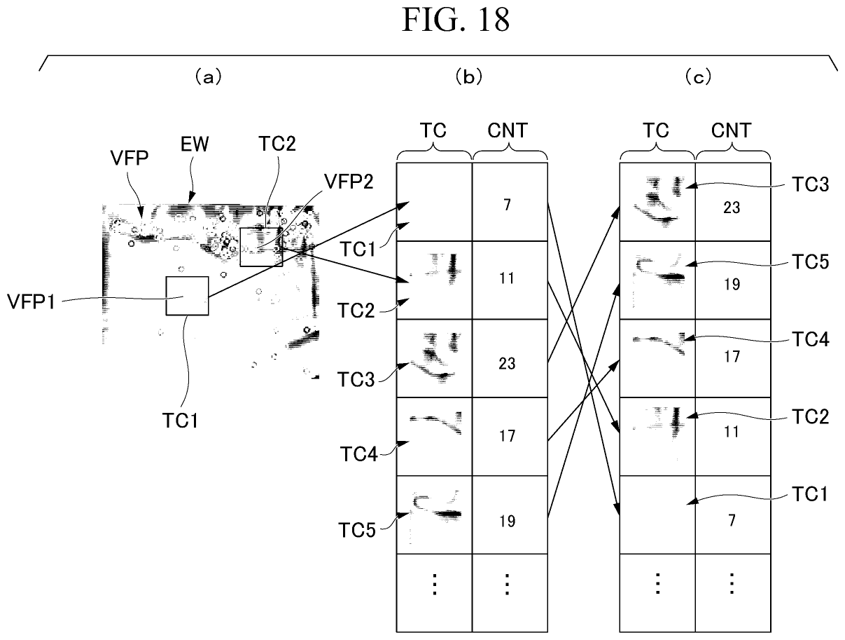

In other words, the candidate region generation unit 130 generates a template candidate region TC for each of the feature points (blood vessel corresponding feature points VFP) selected as the feature points FP each corresponding to the position PV of each blood vessel in the white region EW of the eye, among the feature points FP extracted by the feature point extraction unit 120. A more specific description is given with reference to FIG. 18.

FIG. 18 is a diagram illustrating an example of the template candidate regions TC according to the present embodiment. A plurality of feature points FP are extracted in the white region EW of the eye illustrated in FIG. 18(a). In step S170, the candidate region generation unit 130 generates the template candidate region TC for each feature point FP.

In FIG. 18(a), a case is illustrated in which the candidate region generation unit 130 generates a template candidate region TC1 for a feature point FP1, and a template candidate region TC2 for a feature point FP2, among the plurality of feature points FP. Note that in FIG. 18(a), illustration of template candidate regions TC for other feature points FP is omitted.

Note that in this example, the feature points FP to which the candidate region generation unit 130 refers for generation of the template candidate regions TC are the feature points FP each corresponding to the position of the position PV of each blood vessel (specifically, the blood vessel corresponding feature points VFP) among all the feature points FP.

Next, the candidate region generation unit 130 counts the number CNT of the blood vessel corresponding feature points VFP included in each of the generated template candidate regions TC. In an example illustrated in FIG. 18(b), the candidate region generation unit 130 counts the number CNT of the blood vessel corresponding feature points VFP included in the template candidate region TC1 as “7”. Similarly, the candidate region generation unit 130 counts the number CNT of the blood vessel corresponding feature points VFP for the template candidate region TC2 as “11”, the number CNT of the blood vessel corresponding feature points VFP for the template candidate region TC3 as “23”, the number CNT of the blood vessel corresponding feature points VFP for the template candidate region TC4 as “17”, the number CNT of the blood vessel corresponding feature points VFP for the template candidate region TC5 as “19”, and so on, for each template candidate region TC.

More specifically, the candidate region generation unit 130 cuts out a region of the image of the white region EW of the eye illustrated in FIG. 14 at a position corresponding to a region 50×50 [pixels] around the pixel of each blood vessel corresponding feature point VFP illustrated in FIG. 17, and generates the template candidate region TC by the image of the cut out region. The candidate region generation unit 130 repeats the generation of a template candidate region TC for each blood vessel corresponding feature point VFP.

(Step S180) Returning to FIG. 12, the candidate region generation unit 130 ranks the template candidate regions TC on the basis of the number of feature points FP counted in step S170.

FIG. 18(c) illustrates an example of ranking of the template candidate regions TC ranked by the candidate region generation unit 130.

(Step S190) Returning to FIG. 12, the selection unit 140 selects the template candidate region TC including more of the feature points FP (or the blood vessel corresponding feature points VFP) among the plurality of template candidate regions TC generated by the candidate region generation unit 130, as the template region TP. In other words, the selection unit 140 selects the highly ranked template candidate region TC as the template region TP among the template candidate regions TC ranked by the candidate region generation unit 130.

Here, in a case where ambient light is reflected (reflected back) in the white region EW of the eye, the feature points FP may also be extracted due to the luminance gradient generated by the reflected light. That is, in a case where ambient light is reflected in the white region EW of the eye, the feature points FP may be extracted due to features derived from external disturbances, rather than features derived from the form of the white region EW of the eye. In a case where an image of reflected light is included in a template candidate region TC, the selection unit 140 removes the template candidate region TC including the image of the reflected light so as not to select the template candidate region TC as the template region TP.

More specifically, the selection unit 140 creates a luminance value histogram of the white region EW of the eye, and checks the luminance values in a predetermined range (for example, up to 10%) from the highest for the cumulative frequency of the generated histogram. In a case where a region including greater than or equal to 25 pixels having the luminance values within the predetermined range described above is included in the template candidate region TC, the selection unit 140 determines that ambient light is reflected back in the template candidate region TC.

(Step S200) The selection unit 140 removes the template candidate regions TC which are likely to cause false matching. Specifically, the selection unit 140 selects the template candidate region TC which is less frequently matched with a plurality of regions differing from each other in the eye image IMG among the plurality of template candidate regions TC, as the template region TP.

For example, in a case where there is an image of a blood vessel in a shape close to a straight line in the white region EW of the eye, the region including the image of the blood vessel and not including an image of any of the end points of the blood vessel may be generated as the template candidate region TC. Such a template candidate region TC may also have a high degree of similarity of the image to other regions in the white region EW of the eye. In this case, the template candidate region TC may also match, i.e., mis-match, regions other than the regions that are supposed to be matched.

Thus, the selection unit 140 calculates the number of times that the degree of similarity is greater than 70% as a result of performing template matching on the white region EW of the eye by using the template candidate region TC. Note that the normalized cross-correlation coefficient described below may be used for calculating this degree of similarity.

In the search region (for example, in the white region EW of the eye) by the selection unit 140, there is also a region that corresponds to the template candidate region TC. Therefore, in a case where the selection unit 140 calculates the number of times that the degree of similarity is greater than 70%, the degree of similarity is greater than 70% in at least one time, and in a case where the search region is moved rightward, leftward, upward, or downward by 1 [pixel], the degree of similarity is also greater than 70% in many cases. In other words, in a case where the selection unit 140 calculates the number of times, the number of times that the degree of similarity is greater than 70% may be generally at most five. However, in a case where the number of times that the degree of similarity is greater than 70% is greater than five, it is assumed that false matching occurs. Thus, in a case where the number of times that the degree of similarity is greater than 70% exceeds a predetermined number of times (for example, five times), the selection unit 140 determines that the template candidate region TC is a template candidate region TC that is likely to cause false matching, and removes the template candidate region TC from the selection of the template region TP.

(Step S210) The selection unit 140 selects the template region TP from the template candidate regions TC excluding the template candidate regions TC removed in step S190 and step S200. In other words, the selection unit 140 determines the template region TP.

Returning to FIG. 11, the description is made of the operation of the eye movement measurement system 1.

(Step S20) The acquisition unit 110 acquires the eye image IMG.

### Calculation of Pupil Center Coordinate (Elliptical Fitting)

(Step S30) The measurement unit 150 calculates a pupil center coordinate by known procedures on the basis of the eye image IMG acquired by the acquisition unit 110. Specifically, the measurement unit 150 extracts the region of the pupil image included in the eye image IMG by performing binarization and a labeling process on the eye image IMG. The measurement unit 150 extracts the outline of the pupil from the extracted pupil image, and acquires the convex hull of the outline. The measurement unit 150 calculates the center coordinate of the pupil by performing elliptical fitting on the group of points obtained by the convex hull, for example, by using a least squares method.

Note that the use of elliptical fitting is an example for calculating the center coordinate of the pupil, and the measurement unit 150 may calculate the center coordinate of the pupil by various procedures.

### Calculation of Rotation Angle

The measurement unit 150 tracks the blood vessel image in the white region EW of the eye by using the template region TP described above. Specifically, the measurement unit 150 performs adaptive binarization on the region corresponding to the template region TP of the eye image IMG acquired by the acquisition unit 110, and extracts a blood vessel image indicating the position PV of the blood vessel. The measurement unit 150 selects a region of the eye image IMG with the largest area by performing a labeling process on the eye image IMG after the adaptive binarization process. The normalized cross-correlation coefficient is used for calculating the degree of similarity in the template matching performed by the measurement unit 150. The normalized cross-correlation coefficient R (x, y) is represented by Equations (2) to (4).

$$\begin{matrix}R ( x , y ) = \frac{{Σ}_{{x}^{′} , {y}^{′}} ( {T}^{′} ( {x}^{′} , {y}^{′} ) \cdot {I}^{′} ( x + {x}^{′} , y + {y}^{′} ) )}{\sqrt{{Σ}_{{x}^{′} , {y}^{′}} {{T}^{′} ( {x}^{′} , {y}^{′} )}^{2} \cdot {Σ}_{{x}^{′} , {y}^{′}} {{I}^{′} ( x + {x}^{′} , y + {y}^{′} )}^{2}}} & ( 2 ) \\ {T}^{′} ( {x}^{′} , {y}^{′} ) = τ ( {x}^{′} , {y}^{′} ) - 1 / ( w \cdot h ) \cdot {Σ}_{{x}^{″} , {y}^{″}} T ( {x}^{″} , {y}^{″} ) & ( 3 ) \\ {I}^{′} ( x + {x}^{′} , y + {y}^{′} ) = I ( x + {x}^{′} , y + {y}^{′} ) - 1 / ( w \cdot h ) \cdot {\sum }_{{x}^{″} , {y}^{″}} I ( x + {x}^{″} , y + {y}^{″} ) & ( 4 )\end{matrix}$$

Note that x, y are xy coordinates of the referenced pixel, w is a vertical size of a template image, his a horizontal size of the template image, I is a luminance value in a search image, T is a luminance value of the template image, and R is the result of the calculation of the degree of similarity (x\hat{ }′=0, 1 . . . w−1, y\hat{ }′=0, 1, . . . h−1).

In the template matching described above, (x, y) in which R (x, y) takes the largest value is the coordinates corresponding to the upper left corner of the template region TP described above. The position PV of the blood vessel (the coordinates of the blood vessel image) is defined as the center of the template image. In this case, the coordinates obtained by the template matching are (x+w/2, y+h/2).

### Calculation of Rotation Angle

The measurement unit 150 calculates the rotation angle, based on the result of the template matching by using the template region TP. The measurement unit 150 calculates the rotation angle of the eye from the difference between an angle θi determined from the image of the i-th frame used in the determination of the template region TP and an angle θ (i+t) after t frames from the i-th frame.

As an example, the measurement unit 150 may determine the angle by using a reverse trigonometric function from (x, y) coordinates of two points in a manner similar to that of a simple planar angle calculation, without considering that the eye is spherical for ease of processing. Note that the angle θi calculated from the coordinates of the center of the template region TP with respect to the coordinates of the center of the pupil is expressed by Equation below.

$${θ}_{i} = {tan}^{- 1} \frac{{y}_{vessel} - {y}_{pupil}}{{x}_{vessel} - {x}_{pupil}} \qquad ( 5 )$$

Here, (x_vessel, y_vessel) is the coordinates of the blood vessel image, and (x_pupil, y_pupil) is the pupil center coordinates.

The angle θi determined from the image of the i-th frame used in the determination of the template region TP is set to an eye rotation angle θ [deg.]. The measurement unit 150 calculates the rotation angle from the difference between θi and θ (i+t) determined from the coordinates (x+w/2, y+h/2) of the blood vessel image obtained by the template matching after T frames.

Note that in the template matching described above, the measurement unit 150 may perform the template matching by using the template region TP rotated in advance with the pupil center as the center of rotation.

### MODIFIED EXAMPLE

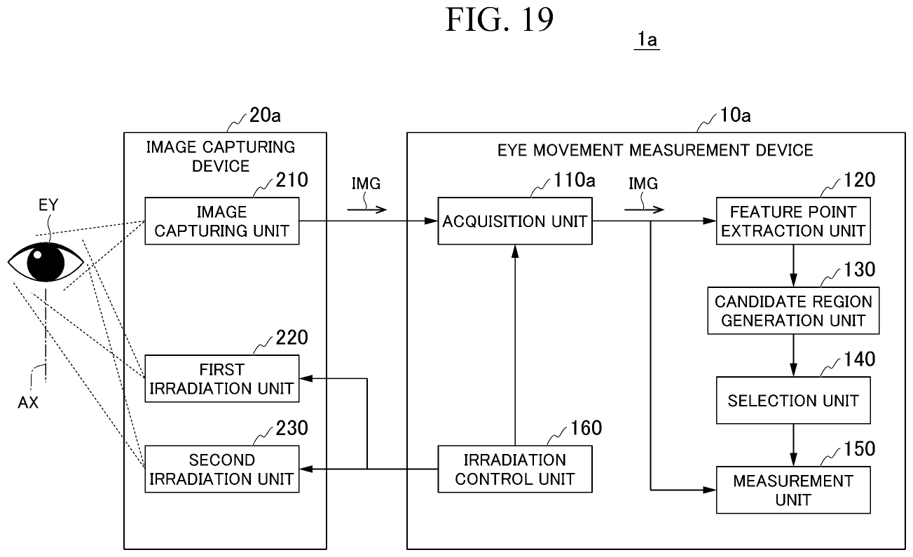

FIG. 19 is a diagram illustrating a modified example of a functional configuration of the eye movement measurement system 1.

An eye movement measurement system 1a according to the present modified example differs from the eye movement measurement system 1 described above in that an eye movement measurement device 10a includes an irradiation control unit 160, and an image capturing device 20a includes a first irradiation unit 220 and a second irradiation unit 230.

The irradiation control unit 160 causes one of the first irradiation unit 220 and the second irradiation unit 230 to emit electromagnetic waves.

The first irradiation unit 220 emits electromagnetic waves to the eye EY of the subject SB. The electromagnetic waves emitted from the first irradiation unit 220 are, for example, visible light in a wavelength region including green light, yellow light, red light, and the like, or infrared rays having a longer wavelength. As an example, the first irradiation unit 220 irradiates the eye EY of the subject SB with electromagnetic waves having a wavelength greater than 495 nanometers. As an example, the first irradiation unit 220 includes a red light emitting diode (LED), and emits red light.

The second irradiation unit 230 irradiates the eye EY of the subject SB with electromagnetic waves having a wavelength shorter than 570 nanometers and shorter than the wavelength of the electromagnetic waves emitted by the first irradiation unit 220. The electromagnetic waves emitted from the second irradiation unit 230 are, for example, visible light in a wavelength region including green light, blue light, purple light, or the like, or ultraviolet rays having a shorter wavelength. As an example, in a case where the first irradiation unit 220 emits electromagnetic waves (for example, green light) having a wavelength of 495 nanometers, the second irradiation unit 230 emits electromagnetic waves having a wavelength shorter than 495 nanometers, for example, electromagnetic waves (for example, blue light) having a wavelength of 450 nanometers. As another example, in a case where the first irradiation unit 220 emits electromagnetic waves (for example, yellow light) having a wavelength of 570 nanometers, the second irradiation unit 230 emits electromagnetic waves having a wavelength shorter than 570 nanometers, for example, electromagnetic waves having a wavelength of 495 nanometers (for example, green light). As an example, the second irradiation unit 230 is provided with a blue LED and emits blue light.

### Alternating Illumination

Here, the irradiation control unit 160 causes one of the first irradiation unit 220 and the second irradiation unit 230 to emit electromagnetic waves. The first irradiation unit 220 emits red light (or electromagnetic waves having a longer wavelength). The second irradiation unit 230 emits blue light (or electromagnetic waves having a shorter wavelength). In the image of the eye EY irradiated with the electromagnetic waves with a long wavelength, the pupil of the eye EY is easy to be visualized. In the image of the eye EY irradiated with the electromagnetic waves with a short wavelength, the blood vessels of the white region EW of the eye EY is easy to be visualized.

In a case where the measurement unit 150 calculates the coordinates of the pupil of the eye EY, the irradiation control unit 160 emits the red light, and in a case where the measurement unit 150 calculates the coordinates of the blood vessels of the eye EY, the irradiation control unit 160 emits the blue light. For example, the irradiation control unit 160 sets the switching period of the wavelength for emission to half the period of the image capturing frame period of the image capturing unit 210.

In the case described above, in a case where the average value of the overall luminance value of the eye image IMG is greater than or equal to a predetermined value (200 in the case of 256 gray scale, for example), the measurement unit 150 detects the pupil center, and in a case where the average value is less than the predetermined value, the measurement unit 150 tracks the positions PV of the blood vessels. The irradiation control unit 160 may output a signal indicating which of electromagnetic waves, between electromagnetic waves having a longer wavelength and electromagnetic waves having a short wavelengths, is emitted, to the image capturing unit 210, the acquisition unit 110, or the measurement unit 150 to synchronize the irradiation wavelength and the captured eye image IMG.

### SUMMARY OF EMBODIMENTS

As described above, the eye movement measurement system 1 according to the present embodiment tracks the movement of the eye image IMG by using the template region TP to measure three-dimensional eye movement. This eye movement measurement system 1 selects the template candidate region TC including more of the feature points FP among the plurality of template candidate regions TC, as the template region TP.

Here, the template candidate region TC including more of the feature points FP has a higher template matching performance than those of template candidate regions TC with fewer feature points FP.

With such a configuration, the eye movement measurement system 1 can achieve the improved tracking performance of the movement of the eye image IMG. In other words, according to the eye movement measurement system 1 according to the present embodiment, the measurement accuracy of the eye movement can be improved.

The eye movement measurement system 1 according to the present embodiment generates the template candidate region TC for each feature point (i.e., blood vessel corresponding feature point VFP) selected as a feature point FP corresponding to the position PV of each blood vessel in the white region EW of the eye, among the extracted feature points FP. Here, the feature points FP extracted from the white region EW of the eye include those derived from an image of the blood vessels of the eye EY and those derived from an image of elements other than the blood vessels (for example, eyelids, eyelashes, dust, and the like). The blood vessels of the eye EY represent movement of the eye EY in an excellent manner since the blood vessels do not change in position relative to the eye EY. On the other hand, elements other than blood vessels do not necessarily represent movement of the eye EY since such elements may change in position relative to the eye EY. Therefore, to improve the tracking performance of the movement of the eye EY, the region including the image of the blood vessel is preferably used as the template region TP, rather than the image of the elements other than a blood vessel is used as the template region TP. That is, in a case where an image of an element other than a blood vessel is used as the template region TP, the tracking performance of the movement of the eye EY is relatively low.

Since a region corresponding to the position PV of each blood vessel is a target region for generating the template candidate region TC, the eye movement measurement system 1 may achieve the improved tracking performance of the movement of the eye EY.

Since a region not corresponding to the position PV of the blood vessel (i.e., a region where the tracking performance of the movement of the eye EY is relatively low) is excluded from the target for generating the template candidate region TC, the number of candidates for the template candidate region TC can be reduced in the eye movement measurement system 1. That is, according to the eye movement measurement system 1, the amount of calculation for selecting the template region TP can be reduced.

In other words, according to the eye movement measurement system 1 configured as described above, the tracking performance of the movement of the eye EY can be improved and the amount of calculation can be reduced in a compatible manner.

The eye movement measurement system 1 according to the present embodiment extracts the feature points FP in the white region EW of the eye by performing a statistical process at least including histogram equalization on the pixel values of the pixels in the white region EW of the eye. Here, in the image of the white of the eye EY, the area of a portion with the base color (white) is relatively large, and the area of a portion with the color (red, through dark red, to black) of the blood vessels to be extracted as the feature point FP is relatively small. In the image of the white of the eye EY, the blood vessels may have a low chroma color, and the contrast of the entire image may be low (weak).

This may make it difficult to extract the image of the blood vessels in a case where the image of the white region EW of the eye is simply binarized. To address this, conventionally, a technique for increasing the contrast of the image by irradiating the eye EY with blue light or the like may be used.

However, according to the eye movement measurement system 1 according to the present embodiment, the pixel values of the respective pixels in the white region EW of the eye are subjected to histogram equalization, so the color of the base color and the color of the blood vessels can be easily distinguished for the white region EW of the eye. That is, according to the eye movement measurement system 1, the extraction performance with respect to the position PV of each blood vessel can be improved, so the tracking performance with respect to the movement of the eye EY can be improved.

The eye movement measurement system 1 according to the present embodiment selects the template candidate region TC having a lower frequency of matching for a plurality of regions differing from each other in the eye image IMG among the plurality of template candidate regions TC, as the template region TP.

Here, the template candidate regions TC include those with relatively high tracking performance and those with relatively low tracking performance with respect to the movement of the eye EY. For example, for a certain template candidate region TC, the template candidate region TC may match a plurality of regions in the white region EW of the eye. In the case of such a template candidate region TC, the tracking performance with respect to the movement of the eye EY is low because it is not possible to determine which region of the plurality of regions matches the template candidate region TC when tracking the movement of the eye EY. In a case where the template candidate region TC matches a single region in the white region EW of the eye and does not match other regions in the white region EW of the eye, the tracking performance with respect to the movement of the eye EY is great. In other words, the tracking performance with respect to the movement of the eye EY is greater when the number of regions which the template candidate region TC matches is smaller.

On the other hand, in a case where the template candidate region TC matches a certain region in the white region EW of the eye, the template candidate region TC may also match regions around the region. Thus, if the condition of selecting the template region TP is limited such that only the template candidate region TC that matches a single region is used, there is a possibility that the choices about the template candidate regions TC may be fewer and tracking performance may be reduced.

The eye movement measurement system 1 according to the present embodiment selects the template region TP, based on the frequency of matching for a plurality of regions. For example, the eye movement measurement system 1 selects the template candidate region TC having a matching frequency of 2 or greater and a predetermined value or less (for example, 5 or less) as the template region TP. In the eye movement measurement system 1 configured in this manner, the number of choices about the template candidate region TC can be inhibited from being reduced, and the improved tracking performance with respect to the movement of the eye EY can be achieved.

The eye movement measurement system 1 according to the present embodiment includes the image capturing unit 210. Since the image capturing device 20 including the image capturing unit 210 and the eye movement measurement device 10 are integrated, the eye movement measurement system 1 can have a simplified wired or wireless communication function for connecting the image capturing device 20 and the eye movement measurement device 10.

The eye movement measurement system 1 includes the first irradiation unit 220, the second irradiation unit 230, and the irradiation control unit 160. The first irradiation unit 220 irradiates the eye EY with electromagnetic waves having a long wavelength (for example, green light, yellow light, red light, or infrared light). Here, in a case where the image capturing unit 210 captures an image of the eye EY irradiated with the electromagnetic waves having a long wavelength, the depiction performance with respect to the pupil of the eye EY is improved in the image generated by the image capturing unit 210. The second irradiation unit 230 irradiates the eye EY with electromagnetic waves having a short wavelength (for example, blue light or ultraviolet light). Here, in a case where the image capturing unit 210 captures an image of the eye EY irradiated with the electromagnetic waves having a short wavelength, the depiction performance with respect to the blood vessels in the white region EW of the eye EY is improved in the image generated by the image capturing unit 210. On the other hand, when the eye EY is irradiated with the electromagnetic waves having a long wavelength and the electromagnetic waves having a short wavelength at the same time, the depiction performance with respect to either (or both) of the pupil of the eye EY and the blood vessels in the white region EW of the eye EY may not be improved.

Since the irradiation control unit 160 of the eye movement measurement system 1 according to the present embodiment causes the electromagnetic waves having a long wavelength and the electromagnetic waves having a short wavelength to be emitted exclusively, both the depiction performance with respect to the pupil of the eye EY and the depiction performance with respect to the blood vessels in the white region EW of the eye EY can be improved.

The embodiments of the present invention have been described in detail above with reference to the drawings, but the specific configuration is not limited to these embodiments, and modifications can be made as appropriate without departing from the spirit of the present invention.

Note that each of the devices described above has a computer inside. The process of each processing of the above-described devices is stored in a computer readable recording medium in the form of a program, and the above processing is performed by a computer reading out and executing the program. Here, the computer readable recording medium refers to a magnetic disk, a magneto-optical disk, a CD-ROM, a DVD-ROM, a semiconductor memory, and the like. The computer program may also be distributed to a computer via a communication line, and the computer that receives this distribution may execute the program.

The program described above may be configured to achieve some of the functions described above.

Furthermore, the functions described above may be achieved in combination with a program already recorded in the computer system, that is, the program may be a so-called differential file (differential program).

### REFERENCE SIGNS LIST

1 Eye movement measurement system
10 Eye movement measurement device
110 Acquisition unit
120 Feature point extraction unit
130 Candidate region generation unit
140 Selection unit
150 Measurement unit
160 Irradiation control unit
20 Image capturing device
210 Image capturing unit
220 First irradiation unit
230 Second irradiation unit

1. An eye movement measurement device comprising:
an acquisition unit configured to acquire an eye image in which an image of an eye of a subject is captured;
a feature point extraction unit configured to extract feature points in a white region of the eye included in the eye image acquired by the acquisition unit;
a candidate region generation unit configured to generate, for each of the feature points extracted by the feature point extraction unit, a template candidate region, the template candidate region being a region including a pixel of each of the feature points, in the eye image;
a selection unit configured to select, as a template region, the template candidate region including more of the feature points among a plurality of the template candidate regions generated by the candidate region generation unit; and
a measurement unit configured to measure a three-dimensional eye movement including at least a rotation angle of the eye of the subject by tracking movement in the eye image acquired by the acquisition unit by using the template region selected by the selection unit.

2. The eye movement measurement device according to claim 1, wherein the candidate region generation unit generates the template candidate region for each of the feature points each selected as a feature point corresponding to a position of a blood vessel in the white region of the eye among the feature points extracted by the feature point extraction unit.

3. The eye movement measurement device according to claim 1, wherein the feature point extraction unit extracts the feature points in the white region of the eye by performing a statistical process including at least histogram equalization on pixel values of the respective pixels in the white region of the eye.

4. The eye movement measurement device according to claim 1, wherein the selection unit selects, as the template region, the template candidate region that less frequently matches a plurality of regions differing from each other in the eye image among a plurality of the template candidate regions.

5. The eye movement measurement device according to claim 1, further comprising an image capturing unit configured to capture an image the eye of the subject to generate the eye image.

6. The eye movement measurement device according to claim 1, further comprising:
a first irradiation unit configured to irradiate the eye of the subject with electromagnetic waves;
a second irradiation unit configured to irradiate the eye of the subject with electromagnetic waves of a wavelength shorter than 570 nanometers and a wavelength shorter than a wavelength of the electromagnetic waves emitted by the first irradiation unit; and
an irradiation control unit configured to cause one of the first irradiation unit and the second irradiation unit to emit electromagnetic waves.

7. An eye movement measurement method comprising:
acquiring an eye image in which an image of an eye of a subject is captured;
extracting feature points in a white region of the eye included in the eye image acquired in the acquiring an eye image;
generating, for each of the feature points extracted in the extracting feature points, a template candidate region, the template candidate region being a region including a pixel of each of the feature points, in the eye image;
selecting, as a template region, the template candidate region including more of the feature points among a plurality of the template candidate regions generated in the generating a template candidate region; and
measuring a three-dimensional eye movement including at least a rotation angle of the eye of the subject by tracking movement in the eye image acquired in the acquiring an eye image by using the template region selected in the selecting the template candidate region.

8. A non-transitory computer readable meeting holding an eye movement measurement program for causing a computer to perform:
acquiring an eye image in which an image of an eye of a subject is captured;
extracting feature points in a white region of the eye included in the eye image acquired in the acquiring an eye image;
generating, for each of the feature points extracted in the extracting feature points, a template candidate region, the template candidate region being a region including a pixel of each of the feature points, in the eye image;
selecting, as a template region, the template candidate region including more of the feature points among a plurality of the template candidate regions generated in the generating a template candidate region; and
measuring a three-dimensional eye movement including at least a rotation angle of the eye of the subject by tracking movement in the eye image acquired in the acquiring an eye image by using the template region selected in the selecting the template candidate region.
